(19) United States
(12) Patent Application Publication
(10) Pub. No.: US 2024/0261106 A1
(43) Pub. Date: Aug. 8, 2024

(54) Implant With Identification Feature For Medical Imaging

(71) Applicant: Stryker European Operations Limited, Carrigtwohill (IE)

(72) Inventor: Eric Tamweber, Tuxedo Park, NY (US)

(21) Appl. No.: 18/105,476

(22) Filed: Feb. 3, 2023

(51) Int. Cl. A61F 2/30; G16H 10/60; G16H 50/70

(52) U.S. Cl. CPC A61F 2/30; G16H 10/60; G16H 50/70; A61F 2002/3008; A61F 2002/3071; A61F 2250/0086

(57) ABSTRACT

The present application relates to apparatus and methods for retrieving medical information using an identification feature on a medical implant in a patient. An identification feature presents as a different color from the remainder of the medical implant under medical imaging, which permits the retrieval of medical information pertaining to the medical implant and/or the patient.

The present disclosure relates to an identification feature on an implant for quickly and easily retrieving medical information during a medical imaging procedure, as well as methods pertaining to same. While discussed largely in connection with use in bone plates, the present disclosure is applicable to other types of medical implants as well.

Medical intervention often results in the placement of medical devices within patients. For instance, it is commonplace to reduce and fix fractures using bone plates. These implanted devices are thereafter viewed under medical imaging procedures that allow a medical professional to monitor/check the device and how the patient anatomy is reacting to the same. These imaging procedures, however, lack the ability to see more than just rudimentary views of the implanted devices.

There is a need to be able to identify medical information directly from an implant within a patient without removing the implant itself, i.e., during medical imaging. Medical professionals cannot rely on receiving accurate information about an implant from their patient, who may not remember the details of the implant or may not even be conscious to share such information during emergency situations. Medical professionals also cannot rely on other institutions to transfer medical information due to the inevitable delay in time, again especially in emergency situations in which medical care must be urgently provided.

Identification of medical information relating to implants is crucial for medical professionals to provide safe and efficient care. This is true not only in emergency situations but also during ongoing care, in which different medical professionals may be monitoring a medical device implanted by a different medical professional at an earlier time. Some implants have associated risks with treatment or diagnostic procedures. For example, patients with a substantially metallic implant may face implant migration when undergoing magnetic resonance imaging (MRI). Even implants that are compatible with diagnostic imaging, such as MRIs, may require reprogramming after the imaging is complete. Therefore, medical professionals need to identify medical information to successfully navigate medical care around potential risks and to plan for any implant-related procedures after medical care is provided.

Thus, it would be desirable to provide a method to identify medical information through an identification feature under medical imaging that does not require removing the implant and that permits quick and easy identification of various attributes pertaining to the implant and/or patient.

### BRIEF SUMMARY OF THE INVENTION

The specific examples herein relate to apparatus and methods for identifying medical information through medical imaging of an implant with an identification feature. The generated image displays the identification feature in a different color than the remainder of the implant. The identification feature in the image is used to identify related medical information.

In one embodiment, the identification feature is text on a surface of the implant that can be read under a medical imaging procedure, such as an X-ray. For instance, the text may be a protrusion or a recession on a surface of the implant. In other embodiments, the identification feature is a barcode or QR code on a surface of the implant. Again, this may be in the form of a protrusion or a recession on a surface of the implant. These codes may be capable of being scanned to identify medical information above and beyond what is capable of being conveyed in the aforementioned text.

One aspect of the present invention is a method of identifying medical information including the steps of imaging an implant implanted in a patient to generate an image of the implant, the implant having an identification feature, locating the identification feature in the image, and identifying the medical information based upon the identification feature.

In accordance with other embodiments of the first aspect, the identification feature may be text on a surface of the implant. The text can be a protrusion or a recession on the surface of the implant. The identification feature may also be a barcode or a QR code on a surface of the implant. Again, these codes can be a protrusion or a recession on the surface of the implant.

The method may further include the step of scanning the barcode or QR code to identify the medical information. The method may include the steps of scanning the barcode or QR code with an image sensor, analyzing the barcode or QR code with a programmed processor, retrieving medical information from a database, displaying the medical information on a display screen and identifying the medical information. The medical information may be a batch number of the implant or a file of the patient.

In certain embodiments, the implant is substantially metallic. In others, the implant is substantially polymeric. In connection with polymeric implants, the identification feature may be radiopaque. For instance, the identification feature may be metallic.

Another aspect of the invention is another method of identifying medical information. This method may include the steps of imaging an implant implanted in a patient to generate an image of the implant, the implant having a barcode or a QR code, locating the barcode or QR code in the image, and scanning the barcode or QR code to identify the medical information.

In embodiments of this second aspect, the barcode or QR code is a protrusion or a recession on a surface of the implant. In other embodiments, the medical information is a batch number of the implant or a file of the patient. The method may also include the steps of analyzing the barcode or QR code with a programmed processor, retrieving medical information from a database, displaying the medical information on a display screen and identifying the medical information.

In other embodiments, the implant is substantially metallic. In others, the implant is substantially polymeric and the identification feature is radiopaque.

### BRIEF DESCRIPTION OF THE DRAWINGS

A more complete appreciation of the subject matter of the present disclosure and the various advantages thereof may be realized by reference to the following detailed description which refers to the accompanying drawings, in which:

FIG. 1 is a perspective view of a bone plate including a protruded identification feature in accordance with one embodiment of the present invention;

FIG. 2 is a side view of the bone plate of FIG. 1;

FIG. 3 is a perspective view of a bone plate including a recessed identification feature in accordance with another embodiment of the present invention;

FIG. 4 is a perspective view of a bone plate including a protruded barcode identification feature in accordance with another embodiment of the presentation invention;

FIG. 5 is a side view of the bone plate of FIG. 4;

FIG. 6 is a perspective view of a bone plate including a protruded QR code in accordance with another embodiment of the present invention;

FIG. 7 is a side view of the bone plate of FIG. 6;

FIG. 8 is a perspective view of a polymeric bone plate including a radiopaque identification feature in accordance with another embodiment of the present invention;

FIG. 9 is a cross-sectional view of the bone plate of FIG. 8 taken along line A-A; and

FIG. 10 depicts the bone plate of FIG. 4 as seen under a medical imaging procedure.

### DETAILED DESCRIPTION

As used herein unless stated otherwise, the term “anterior” means toward the front part of the body, and the term “posterior” means toward the back part of the body. When referring to specific directions in the following description, the terms “proximal” and “distal” are to be understood in regard to the device's orientation and position during exemplary application to human body. Thus, the term “proximal” means closer to the operator or in a direction toward the operator, and the term “distal” means more distant from the operator or in a direction away from the operator. In addition, the terms “about,” “generally.” and “substantially” are intended to mean that deviations from absolute are included within the scope of the term so modified.

Turning now to the figures, a bone plate is shown and will be discussed below. The particular examples shown permit the retrieval of medical information using identification features on a bone plate that can be viewed under medical imaging. However, the various examples of the bone plate disclosed below are not so limited to just the specific designs and uses disclosed herein. For instance, while shown generally as a small fragment bone plate, the present invention has applicability for other types of bone plates (e.g., for long bones, for craniomaxillofacial, or the like). Moreover, it is contemplated to incorporate the present invention in connection with other medical implants (e.g., orthopedic implants like knee and hip implants and bone nails), medical devices for use in the cardiovascular system (e.g., stents, pacemakers, catheters, or the like), and any other devices to be implanted in the body.

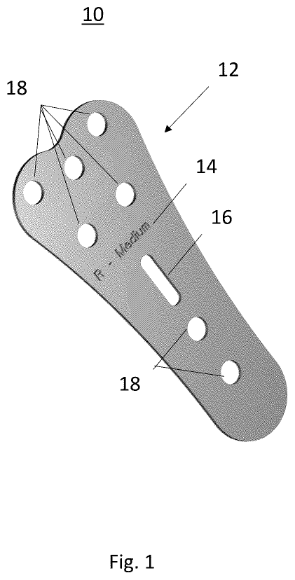

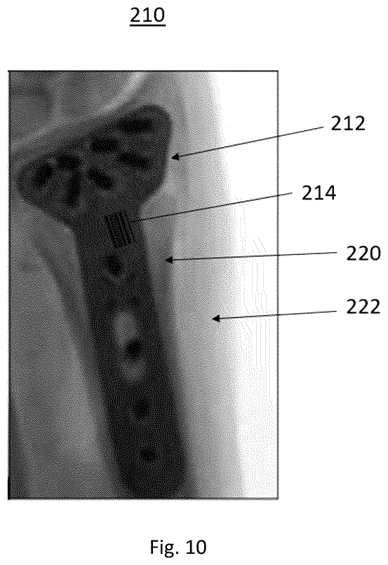

As shown in FIG. 1, in accordance with one embodiment of the present invention, a bone plate 10 includes a slot 16 and multiple bone screw holes 18. Slot 16 is located at the middle of the bone plate 10, while five holes 18 are located on the anterior end of bone plate 10 and three holes 18 are located on the posterior end of bone plate 10. Bone plate 10 further includes an identification feature 14, which is located between slot 16 and holes 18 on the anterior end of the bone plate 10. Identification feature 14 is depicted as text protruding from the surface of the bone plate 10 pertaining to the size and shape of the plate. Under medical imaging, the identification feature 14 may be a different color than the base 12, as is best shown in FIG. 10.

Although shown as a specifically sized and shaped plate, it is to be understood that plate 10 can be any shape or size for use in connection with different bones. Moreover, the specific configurations of slot 16 and holes 18 can vary depending upon the type of implant/plate being utilized. Bone plate 10 (and any other implant in accordance with the present invention) can be made of any material suitable for implanting into a human body, including but not limited to polymer materials such as PEEK and metallic materials, such as stainless steel or titanium.

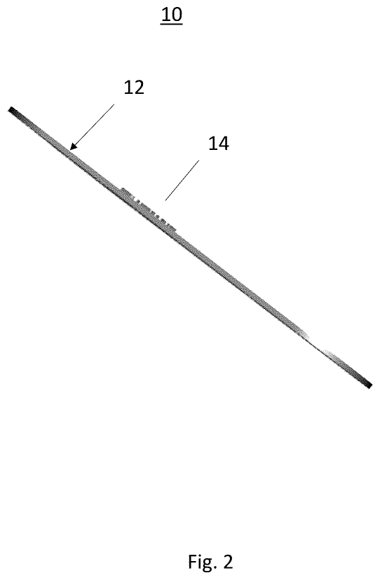

FIG. 2 is a side view of bone plate 10 of FIG. 1. As shown, the text of identification feature 14 protrudes a certain distance from the surface of bone plate 10. This protrusion of the text permits such to be read under a medical imaging process, such as an X-ray. It is to be understood that the amount identification feature 14 protrudes can vary and, in certain instances, less protrusion may be desired to preclude irritation of underlying tissue. Moreover, although shown as text, it is to be understood that identification feature 14 can be any other type of indicia, certain of which will be discussed below.

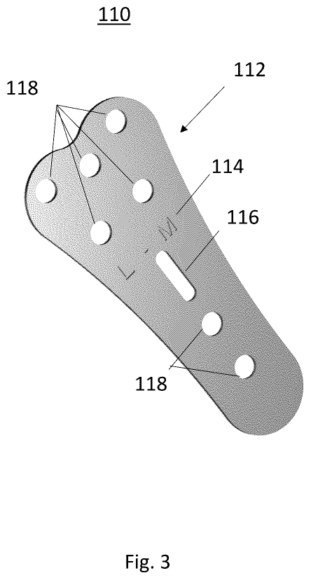

FIG. 3 depicts a bone plate 110 in accordance with another embodiment of the present invention. This plate includes similar features to that of plate 10, with like reference numerals being utilized for like features, but within the 100-series of numbers. For instance, plate 110 includes base 112, slot 116 and holes 118. However, plate 110 differs in that identification feature 114 is depicted as text recessing from the surface of the bone plate 110 pertaining to the size and shape of the plate. Again, identification feature 114 may be read under medical imaging.

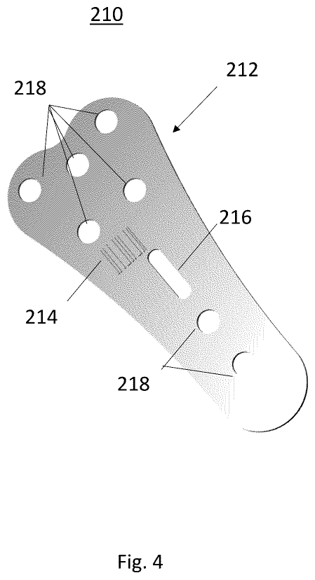

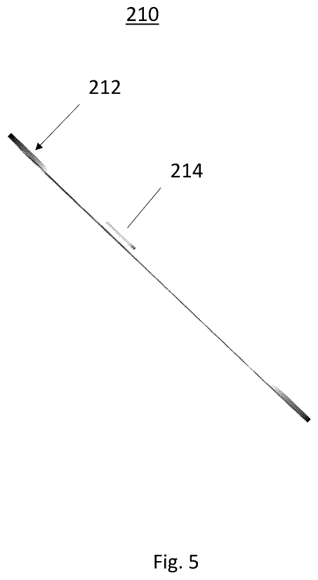

FIG. 4 depicts a bone plate 210 in accordance with another embodiment of the present invention. This plate includes similar features to that of plates 10 and 110, with like reference numerals being utilized for like features but within the 200-series of numbers. Here, plate 210 differs in that identification feature 214 is depicted as a bar code protruding from the surface of the bone plate 210 (see FIG. 5) pertaining to information associated with plate 210. This information, like in the foregoing plates, can pertain to the size and shape of the plate. However, it is contemplated to have the bar code associated with other information, like patient information or surgery information. Although shown protruding from plate 210, identification feature 214 can be recessed like in plate 110.

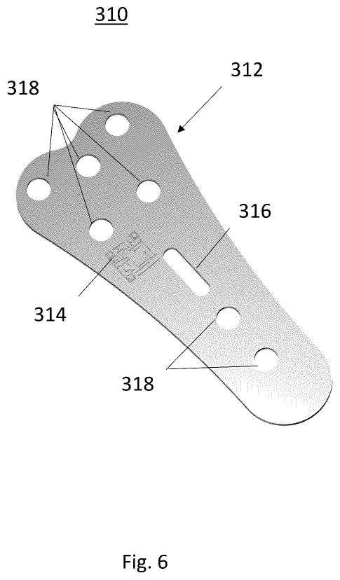

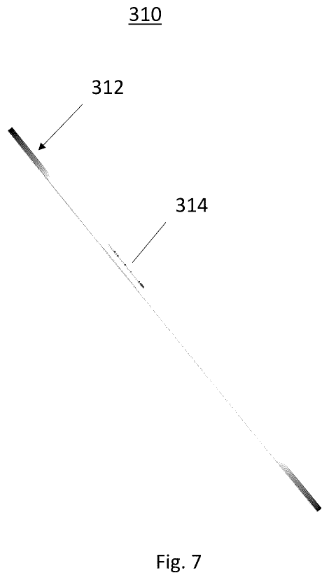

FIG. 6 depicts a bone plate 310 in accordance with another embodiment of the present invention. This plate includes similar features to that of plates 10, 110 and 210, with like reference numerals being utilized for like features but within the 300-series of numbers. Here, plate 310 differs in that identification feature 314 is depicted as a QR code protruding from the surface of the bone plate 310 (see FIG. 7) pertaining information associated with plate 310. This information, like in the foregoing plates, can pertain to the size and shape of the plate. However, it is contemplated to have the QR code associated with other information, like patient information or surgery information. Although shown protruding from plate 310, identification feature 314 can be recessed like in plate 110.

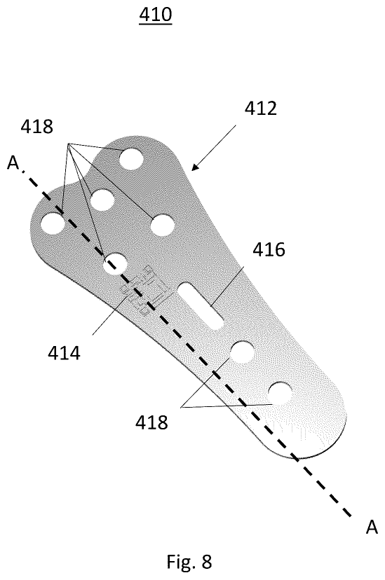

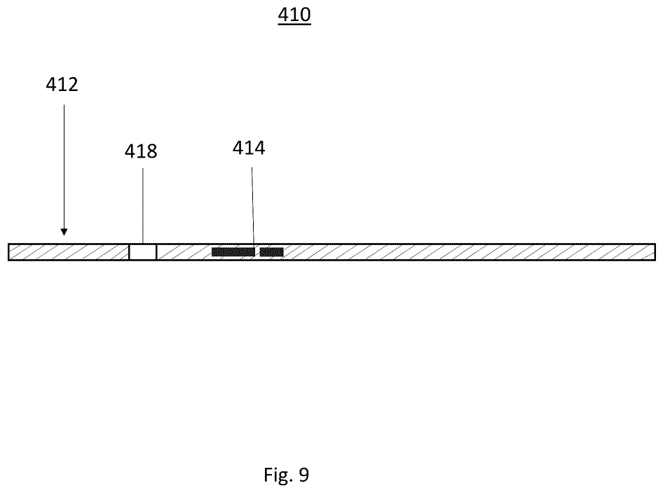

FIG. 8 depicts a bone plate 410 in accordance with another embodiment of the present invention. This plate includes similar features to that of plates 10, 110, 210 and 310, with like reference numerals being utilized for like features but within the 400-series of numbers. Here, plate 410 differs in that it is formed of substantially polymeric material. Identification feature 414 is formed of a radiopaque material, such as tantalum or other metallic materials. A cross-sectional view of plate 410 taken along line A-A of FIG. 8 is shown in FIG. 9.

Each of the identification features of the foregoing embodiments are designed to show up clearly in a medical image such as an X-ray. For instance, as shown in FIG. 10, identification feature 214 features prominently in the X-ray image as being a different color from the remainder of plate 210, bone 220, and soft tissue 222. To be clear, this may include being a different shade of the same color. This permits a surgeon or other medical professional to quickly and easily garner information from a simple medical imaging procedure. This includes information that may be linked to the bar and QR codes of plates 210 and 310. For instance, these codes may link to a patient file or to surgery notes. Thus, from a simple medical image a large amount of information can be garnered without any significant intervention on the patient.

Although the medical image shown in FIG. 10 depicts identification feature 214 in a lighter shade of gray and base 212 in a darker shade of gray, it is to be understood that identification feature 214 can be any color that is different than base 212 in the medical image. For instance, the colors of identification feature 214 and base 212 in a medical image can differ in hue, shade, saturation, brightness, or gloss. With respect to plate 410, identification feature 414 may be the only feature of the plate that shows in any great detail in a medical image. Given the polymeric nature of body 412, very little of that material will show up on a medical image such as an X-ray. Of course, it is contemplated to vary the materials utilized in plates according to the present invention in order to in turn vary the amount different portions show on X-rays or the like.

Although the medical image shown as an x-radiation image, it is to be understood that medical imaging can include any imaging method of an implant inside of a patient. For instance, the medical imaging may also be captured through computerized tomography (CT) scans, positron emission tomography (PET) scans, magnetic resonance imaging (MRI) scans, and ultrasound scans.

In accordance with the present invention, identification feature 14 can provide information about the implant. For instance, the information may include the specific model, size, manufacturer, date of manufacture, brand, composition, and expiration date. Identification feature 14 may also provide information necessary for the removal of the implant, such as the type of instrumentation required for removal.

As noted above, identification feature 14 can also provide information about the patient. For instance, the information may include the patient's general medical records, including their name, address, medical history, treating physicians, diagnoses, and current or past medications. The information may also include records of the implantation of the implant, such as images of the implant operation, date of implantation, and any medical procedures associated with the implant.

Identification feature 14 on a substantially metallic bone plate 10 may be manufactured by various processes, including 3D printing or milling, and identification feature 14 on a substantially polymeric bone plate 10 may be manufactured by processes such as injection molding or overmolding the polymeric material around feature 14.

The foregoing barcode and QR code examples are capable of being scanned by a two-dimensional digital image sensor, such as a smart phone, tablet, or computer. For instance, feature 14 as a QR code is preferably capable of being analyzed by a programmed processor to convert the squares throughout the QR code to binary numbers, which are then converted to medical information. The medical information may be retrieved from a database comprising implant or patient information. The medical information may be portrayed on a display screen, such as a smart phone, tablet, computer or the like. Again, this can very useful to quickly garner information in a simple scan setting.

It is to be understood that the disclosure set forth herein includes any possible combinations of the particular features set forth above, whether specifically disclosed herein or not. For example, where a particular feature is disclosed in the context of a particular aspect, arrangement, configuration, or arrangement, that feature can also be used, to the extent possible, in combination with and/or in the context of other particular aspects, arrangements, configurations, and arrangements of the technology, and in the technology generally.

Furthermore, although the technology herein has been described with reference to particular features, it is to be understood that these features are merely illustrative of the principles and applications of the present technology. It is therefore to be understood that numerous modifications, including changes in the sizes of the various features described herein, may be made to the illustrative arrangements and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A method of identifying medical information comprising the steps of:
imaging an implant implanted in a patient to generate an image of the implant, the implant having an identification feature;
locating the identification feature in the image, and
identifying the medical information based upon the identification feature.

2. The method of identifying medical information of claim 1, wherein the identification feature is text on a surface of the implant.

3. The method of identifying medical information of claim 2, wherein the text is a protrusion or a recession on the surface of the implant.

4. The method of identifying medical information of claim 1, wherein the identification feature is a barcode or a QR code on a surface of the implant.

5. The method of identifying medical information of claim 4, wherein the identification feature is a protrusion or a recession on the surface of the implant.

6. The method of identifying medical information of claim 5, further comprising the step of scanning the barcode or QR code to identify the medical information.

7. The method of identifying medical information of claim 6, further comprising the steps of:
scanning the barcode or QR code with an image sensor;
analyzing the barcode or QR code with a programmed processor;
retrieving medical information from a database;
displaying the medical information on a display screen; and
identifying the medical information.

8. The method of identifying medical information of claim 1, wherein the medical information is a batch number of the implant.

9. The method of identifying medical information of claim 1, wherein the medical information is a file of the patient.

10. The method of identifying medical information of claim 1, wherein the implant is substantially metallic.

11. The method of identifying medical information of claim 1, wherein the implant is substantially polymeric.

12. The method of identifying medical information of claim 11, wherein the identification feature is radiopaque.

13. The method of identifying medical information of claim 12, wherein the identification feature is metallic.

14. A method of identifying medical information comprising the steps of:
imaging an implant implanted in a patient to generate an image of the implant, the implant having a barcode or a QR code;
locating the barcode or QR code in the image, and
scanning the barcode or QR code to identify the medical information.

15. The method of identifying medical information of claim 14, wherein the barcode or QR code is a protrusion or a recession on a surface of the implant.

16. The method of identifying medical information of claim 14, wherein the medical information is a batch number of the implant.

17. The method of identifying medical information of claim 14, wherein the medical information is a file of the patient.

18. The method of identifying medical information of claim 14, further comprising the steps of:
analyzing the barcode or QR code with a programmed processor,
retrieving medical information from a database;
displaying the medical information on a display screen; and
identifying the medical information.

19. The method of identifying medical information of claim 14, wherein the implant is substantially metallic.

20. The method of identifying medical information of claim 14, wherein the implant is substantially polymeric and the identification feature is radiopaque.
